(19) United States
(12) Patent Application Publication
(10) Pub. No.: US 2007/0056590 A1
(43) Pub. Date: Mar. 15, 2007

(54) Holder for nasal cannula

(76) Inventor: Ivan A. Wolfson, Highland Park, IL (US)

Correspondence Address: BRINKS HOFER GILSON & LIONE, P.O. BOX 10395, CHICAGO, IL, 60610, US

(21) Appl. No.: 11/226,759

(22) Filed: Sep. 14, 2005

(51) Int. Cl. A61M 15/08; A62B 7/00

(52) U.S. Cl. 128/207.18

(57) ABSTRACT

A nasal cannula holding system includes a support band adapted to be disposed on the head of a patient. A first fastener is adapted to be disposed adjacent the right ear of the patient and connected to the support band. A second fastener is adapted to be disposed adjacent the left ear of the patient and connected to the support band. A nasal cannula includes a nose piece and right and left cannula tubes and is adapted to be used on the nose of a patient. The right cannula tube is slidably connected to the right fastener and the left cannula tube is slidably connected to the left fastener.

### FIELD OF THE INVENTION

The present invention relates to a nasal cannula holding system.

### BACKGROUND

Nasal cannulas are used to transport oxygen or other gases for inhalation to the nose of a patient. The nasal cannula has two tubes which fit into the nostrils of a patient. It is desirable to securely hold the cannula in place under the nostrils of the wearer. Typically, a nosepiece is partially inserted into the nostrils, and the cannula tubes extend from the nosepiece back over the top of the ears, around the ears and down together along the front of the chest to the oxygen supply. However, this arrangement of the cannula often causes discomfort for the wearer. The oxygen tubes can chafe the patient's skin on the cheeks and over the ears. Also, the cannula often may be easily dislodged during sleeping or movement, reducing its effectiveness. Also, because the cannula is uncomfortable, some patients are more likely to purposely remove it or to leave it off when it becomes dislodged.

### SUMMARY

A nasal cannula holding system is provided for securing a nasal cannula to the head of a patient. In various aspects, the nasal cannula holding system may reduce chafing and other irritation and may prevent the nasal cannula from being dislodged from the patient's nostrils.

In one aspect, the nasal cannula holding system includes a support band adapted to be disposed on the head of a patient. A first fastener is adapted to be disposed adjacent the right ear of the patient and connected to the support band. A second fastener is adapted to be disposed adjacent the left ear of the patient and connected to the support band. A nasal cannula includes a nose piece and right and left cannula tubes and is adapted to be used on the nose of a patient. The right cannula tube is connected to the right fastener and the left cannula tube is connected to the left fastener.

In another aspect, method of maintaining the position of a nasal cannula relative to the head of a patient includes providing a support band and positioning the support band on the head of a patient. A first fastener is attached to the support band on the right side of the patient's head. A second fastener is attached to the support band on the left side of the patient's head. A nasal cannula including a nose piece and right and left cannula tubes is provided. The nose piece into the nostrils of the patient and the right and left cannula tubes to the respective sides of the patient's head. The right cannula tube is connected to the first fastener and the left cannula tube is connected to the second fastener to secure the nasal cannula in place.

The foregoing paragraphs have been provided by way of general introduction, and are not intended to limit the scope of the following claims. The presently preferred embodiments, together with further advantages, will be best understood by reference to the following detailed description taken in conjunction with the accompanying drawings.

### BRIEF DESCRIPTION OF THE DRAWINGS

The invention is described with reference to the drawings in which like elements are referred to by like numerals. The relationship and functioning of the various elements of this invention are better understood by the following detailed description. However, the embodiments of this invention as described below are by way of example only, and the invention is not limited to the embodiments illustrated in the drawings.

FIG. 1 shows one embodiment of a nasal cannula holding system.

FIG. 2 shows a side view of a patient's head with one embodiment of a nasal cannula holding system disposed on the patient's head.

FIG. 3 shows a front view of a patient's head with one embodiment of a nasal cannula holding system disposed on the patient's head.

FIG. 4 shows a side view of a patient's head with another embodiment of a nasal cannula holding system disposed on the patient's head.

FIG. 5 shows a side view of a patient's head with another embodiment of a nasal cannula holding system disposed on the patient's head.

FIG. 6 shows a side view of a patient's head with another embodiment of a nasal cannula holding system disposed on the patient's head.

FIG. 7 shows another embodiment of a nasal cannula holding system.

### DETAILED DESCRIPTION

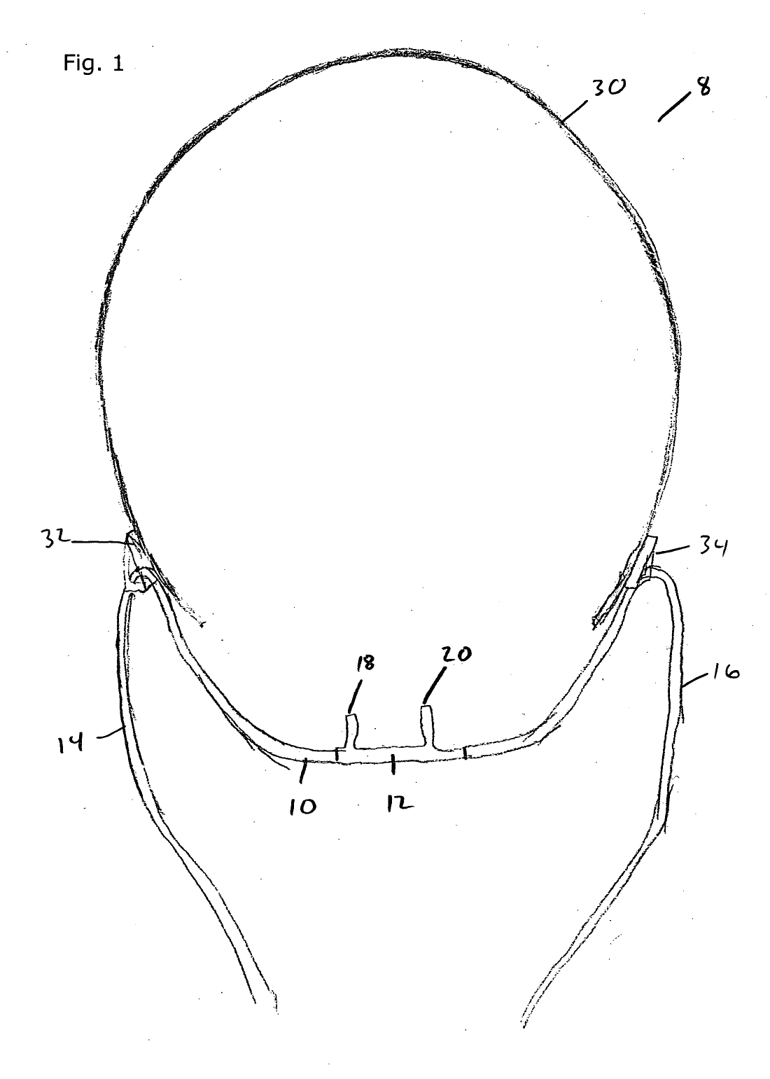

A nasal cannula holding system is disclosed. A nasal cannula is a device used to transport oxygen or other gases for inhalation to the nose of a patient. As shown in FIG. 1, the nasal cannula 10 includes a nose piece 12 and right and left cannula tubes 14, 16 and is adapted to be used on the nose of a patient. The nose piece includes left and right nose tubes 20, 18. The nasal cannula holding system 8 includes a support band 30 adapted to be disposed around the head of a patient and first and second fasteners 32, 34. The first fastener 32 is adapted to be disposed adjacent the right ear of the patient and connected to the support band 30. The second fastener 34 is adapted to be disposed adjacent the left ear of the patient and connected to the support band 30. The right cannula tube 14 is connected to the right fastener 32 and the left cannula tube 16 is connected to the left fastener 34.

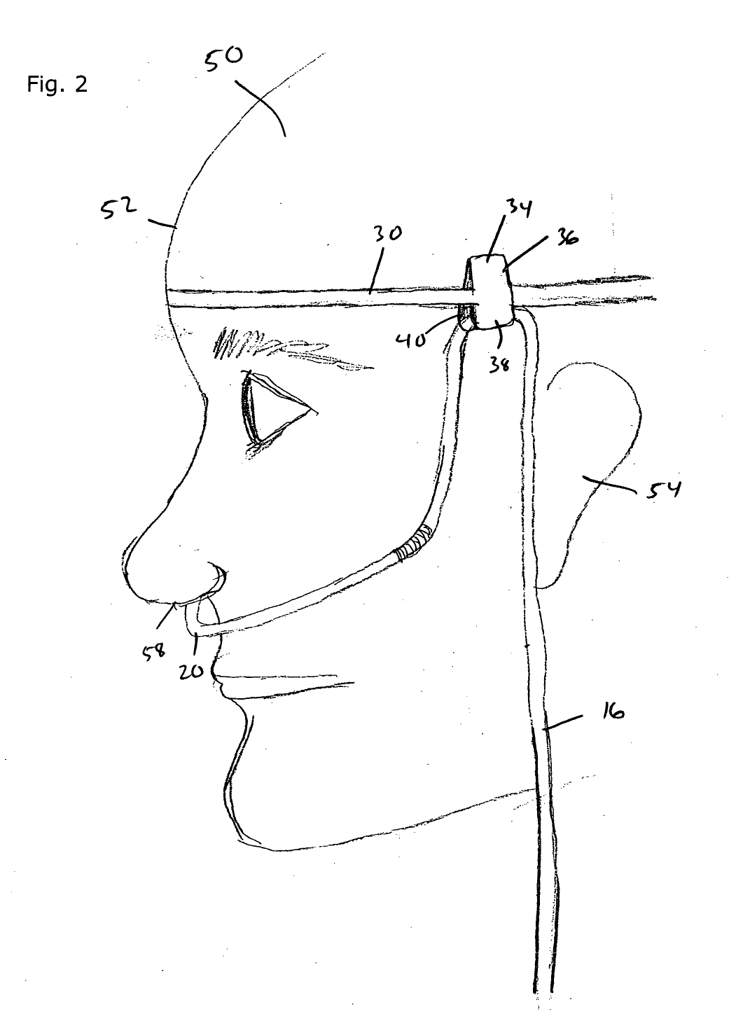

FIG. 2 shows a side view of a patient's head with one embodiment of a nasal cannula holding system disposed on the patient's head 50. The support band 30 may be disposed around the patient's forehead and around the head above the ears 54. A variety of fastener configurations are possible. The fasteners 32, 34 may be either fixedly or slidably attached to the support band 30. When the fasteners 32, 34 are slidably attached, their location may be easily adjusted for patient comfort and effectiveness. In one embodiment, shown in FIG. 2, the fastener 24 includes a strap 36 with a first end 38 and a second end 40, the first end 32 releasably fastened to the second end 34 to form a loop. The ends 38, 40 may be connected together by any suitable method, such as by a hook-and-loop fabric connection (e.g. Velcro TM) or by a snap fit connection. The loop of each fastener 32, 34 is disposed around the respective cannula tube 14, 16. The cannula tube 16 is disposed forward of the patient's ear 54, runs through the fastener 34, and then runs from the fastener 34 to the patient's nostril 58. The nasal cannula holding system both keeps the nasal cannula 10 in place on the nose of a patient and also keeps the tubes 14, 16 away from the patient's skin to prevent chafing and other irritation of the skin.

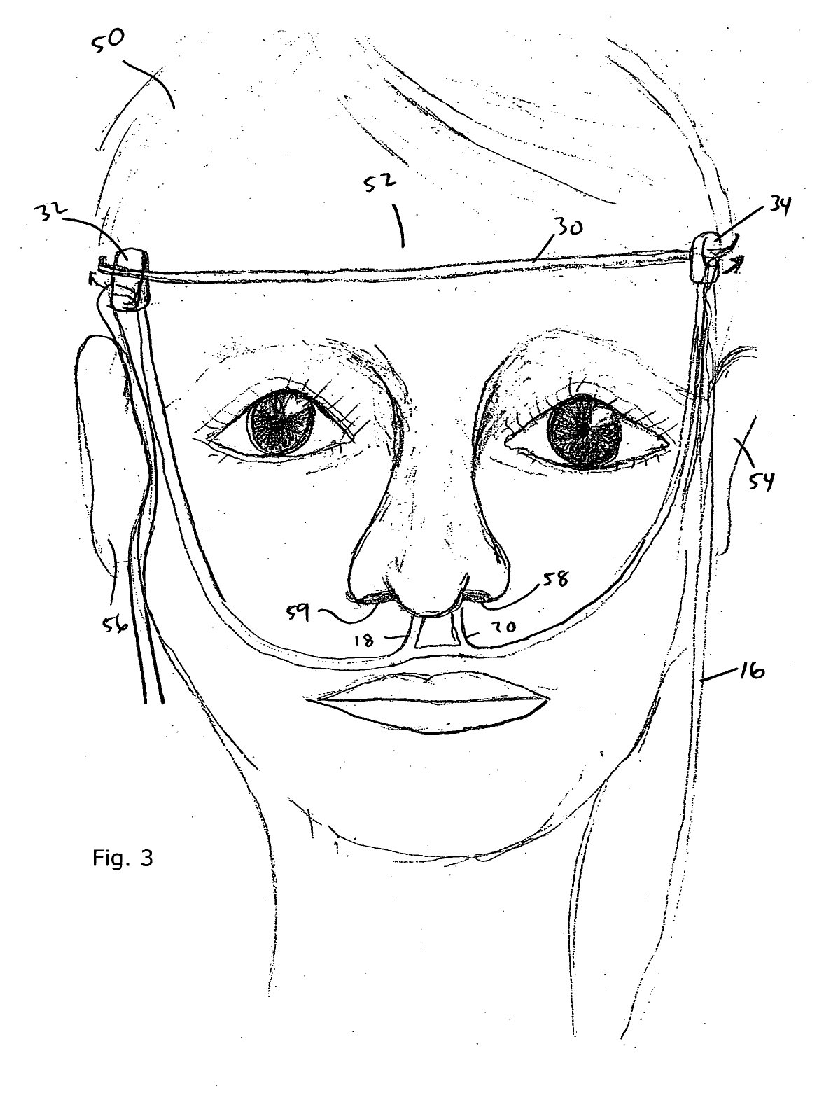

FIG. 3 is a front view of the nasal cannula holding system. The cannula tubes 14, 16 are disposed forward of the patient's ears 54, 56, run through the fasteners 32, 34, and then run from the fasteners 32, 34 to the patient's nostrils 59, 58. The nose tubes 18, 20 are disposed in the patient's nostrils 59, 58.

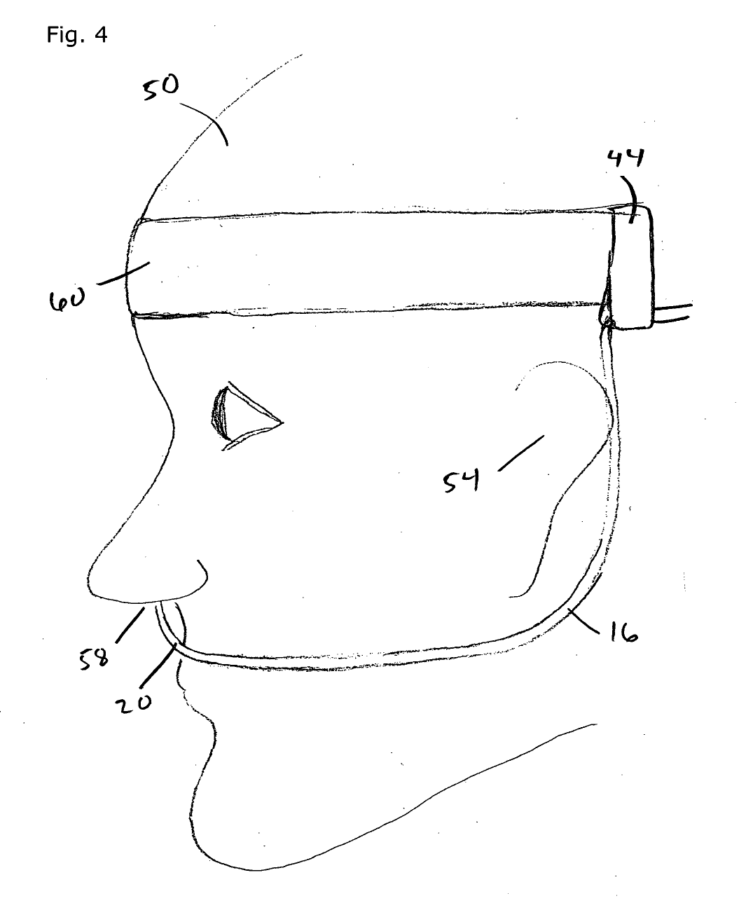

FIG. 4 shows the nasal cannula holding system with an alternative support band 60. The support band 60 is wider than support band 30 and may be constructed similarly to a conventional headband or sweatband. The wider support 60 may be more comfortable for some patients. The fastener 44 may be permanently attached to the support band 60 (e.g. by sewing or adhesive). The fastener 44 may also be slidably attached to the support band 60, for example if fastener 44 is fashioned as a loop. FIG. 4 also shows an alternative arrangement of the cannula tube where the cannula tube 16 is disposed behind the patient's ear 54. The cannula tube 16 runs through the fasteners 44 behind the patient's ear 54, and to the patient's nostril 58.

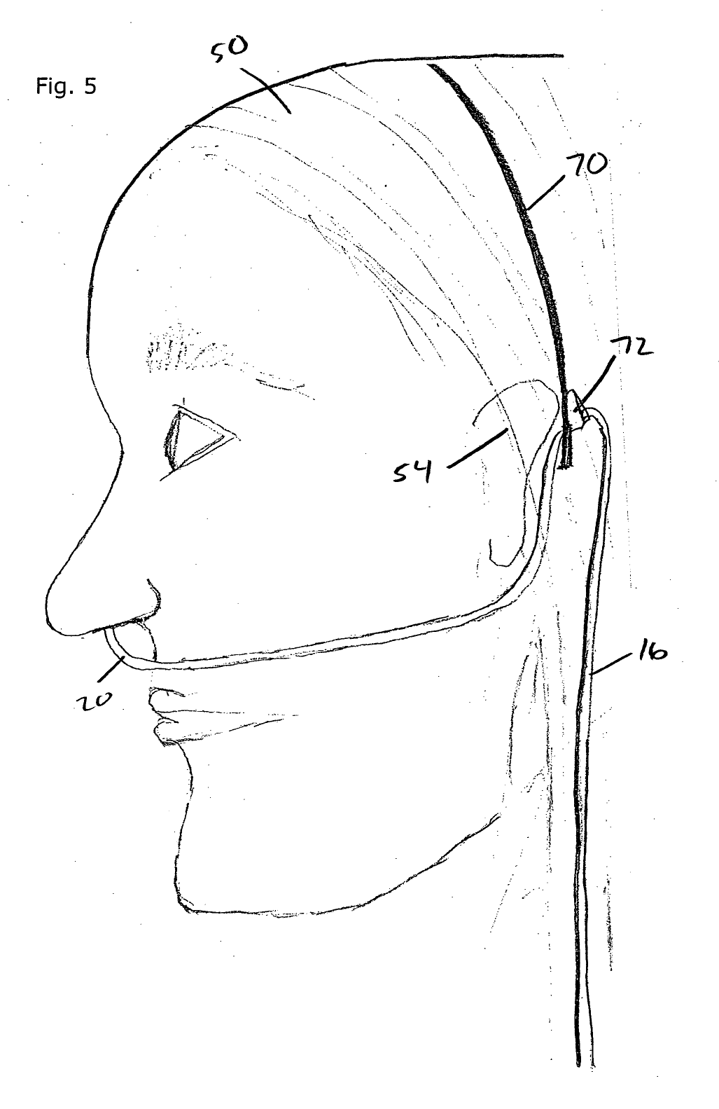

The support band 30 may be disposed around the head of a patient in a variety of configurations. In one embodiment, the support band 30 is disposed around the forehead 52 of the patient and around the sides of the patient's head above the ears, as shown in FIGS. 2, 3, and 4. In another embodiment, a support band 70 is disposed around the top of the patient's head, as shown in FIG. 5. The support band 70 runs on the top of a patient's head 50 and behind the ears 54, much like a pair of head phones. The support band 70 is preferably inwardly biased in order to stay secured to the patient's head 50. The cannula tube 16 is secured to the support band 70 by fastener 72, which may be any of the fastener designs disclosed herein.

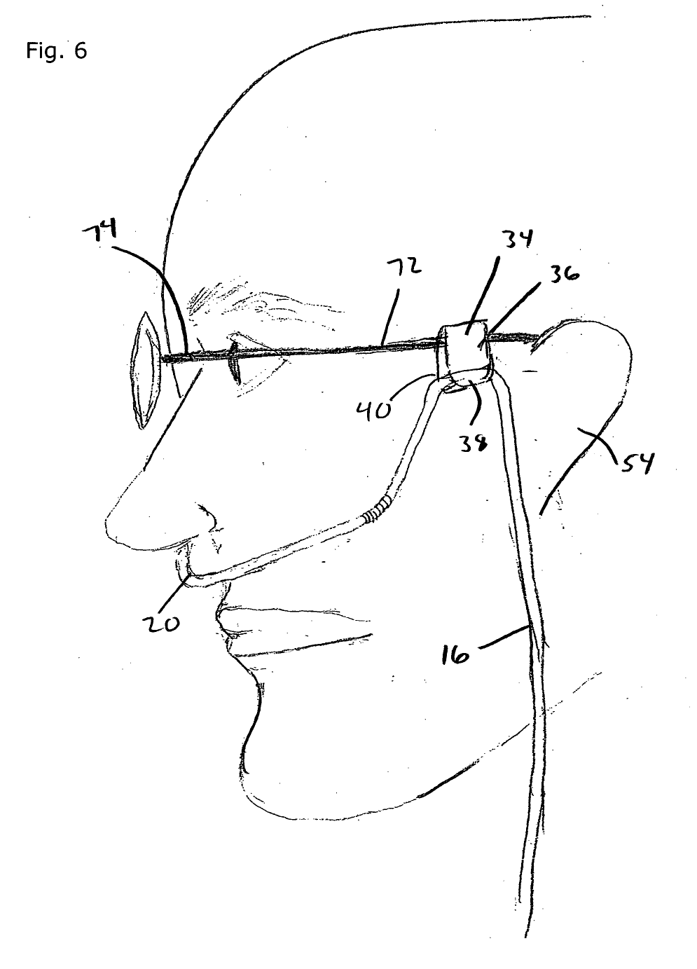

In yet another embodiment, the loops are supported by the temples 72 of the patient's glasses 72, as shown in FIG. 6. The fastener 34 is attached to the temple 72. If the fastener 34 is slidably attached, its location may be easily adjusted along the temple 72 for patient comfort and effectiveness. The fastener 34 may include a strap 36 with a first end 38 and a second end 40, the first end 32 releasably fastened to the second end 34 to form a loop. The ends 38, 40 may be connected together by any suitable method. The loop of the fastener 34 is disposed around the cannula tube 16.

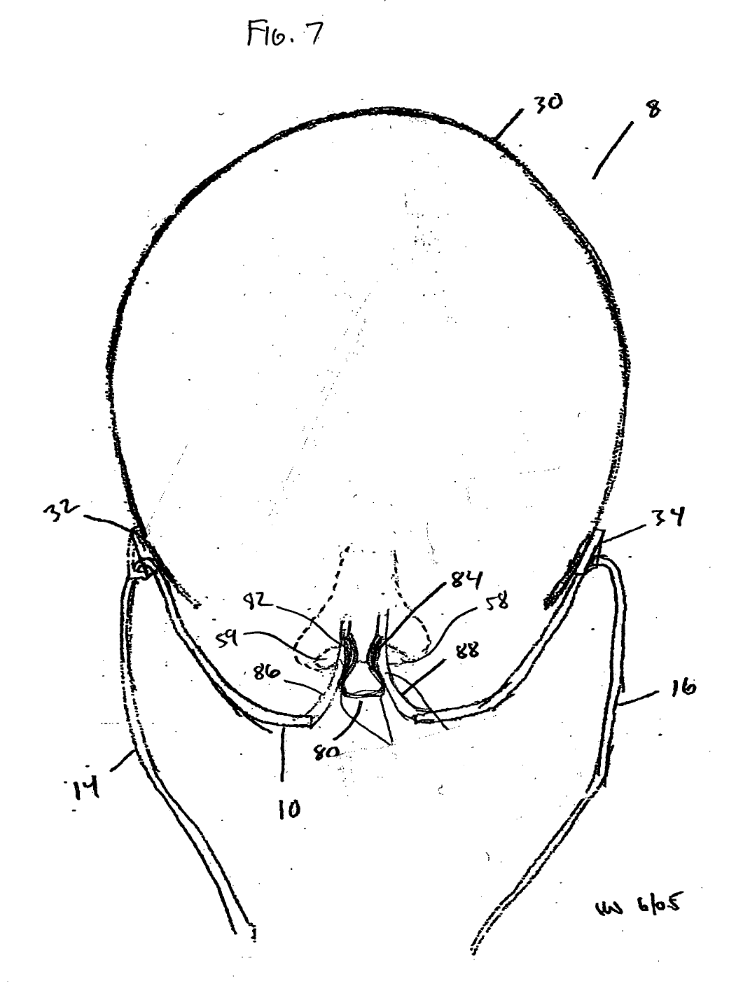

FIG. 7 shows another embodiment of a nasal cannula holding system. In this embodiment, the nasal cannula holding system further includes a nose clamp 80. The nose clamp 80 includes two clips 82, 84. Clips 82, 84 are inwardly biased so that they clamp onto the patient's nose adjacent the septum. The left and right nose tubes 88, 86 are connected to the nose clips 84, 82 respectively. This arrangement ensures a secure fit of the nose tubes 88, 86 to the patient's nose. The nose clamp may be used by first spreading each clip 82, 84 apart and then inserting each clip 82, 84 into the respective nostril 59, 58. The biasing force of the nose clamp 80 ensures that the nose tubes 86, 88 remain in the patient's nostrils.

As best seen in FIGS. 2 and 3, a method of maintaining the position of a nasal cannula 10 relative to the head of a patient includes providing the support band 30 and positioning the support band 30 around the head of a patient 50. The first fastener 32 is attached to the support band 30 on the right side of the patient's head, and a second fastener 34 is attached to the support band 30 on the left side of the patient's head. The fasteners 32, 34 may also be provided pre-attached to the support band 30. A nasal cannula nose piece 20 is disposed into the nostrils of the patient. The right and left cannula tubes 14, 16 are disposed to the respective sides of the patient's head. The right cannula tube 14 is attached to the first fastener 32 and the left cannula tube 16 is attached to the second fastener 34 to secure the nasal cannula in place.

The fasteners may be disposed in a variety of locations on a patient's head. In one embodiment, as shown in FIG. 2, the fasteners 34 are disposed forward of the ears of the patient. However, the fasteners may also be disposed behind the ears of a patient, as shown in FIG. 4. In both cases, the fasteners position the cannula tubes 14, 16 away from the skin of the patient.

The components of the cannula holding system may be made of any suitable material. The support band 30, 60 is preferably made of a soft fabric material. The support band 30, 60 may also be made of an elastic material in order to fit securely on a patient's head. The fasteners 32, 34 are preferably made of a fabric material in order to form a loop. The support band 70 shown in FIG. 5 may be made of a more rigid material such as plastic which is capable of providing a biasing force.

The embodiments described above and shown herein are illustrative and not restrictive. The scope of the invention is indicated by the claims rather than by the foregoing description and attached drawings. The invention may be embodied in other specific forms without departing from the spirit of the invention. Accordingly, these and any other changes which come within the scope of the claims are intended to be embraced therein.

What is claimed is:

1. A nasal cannula holding system comprising:
a support band adapted to be disposed on the head of a patient;
a first fastener connected to the support band and adapted to be disposed adjacent the right ear of the patient;
a second fastener connected to the support band and adapted to be disposed adjacent the left ear of the patient; and
a nasal cannula comprising a nose piece and right and left cannula tubes and adapted to be used on the nose of a patient, wherein the right cannula tube is slidably connected to the right fastener and the left cannula tube is slidably connected to the left fastener.

2. The nasal cannula holding system of claim 1 wherein each fastener comprises a strap with a first end and a second end, the first end releasably fastened to the second end to form a loop.

3. The nasal cannula holding system of claim 2 wherein the first end is attached to the second end by a hook-and-loop fabric connection.

4. The nasal cannula holding system of claim 2 wherein the first end is attached to the second end by a snap fit connection.

5. The oxygen tube holding system of claim 2 wherein the loop of each fastener is disposed around the cannula tube.

6. The nasal cannula holding system of claim 1 wherein each fastener is fixedly attached to the support band.

7. The nasal cannula holding system of claim 1 wherein each fastener is slidably attached to the support band.

8. The nasal cannula holding system of claim 1 wherein the support band is adapted to be disposed around the forehead of the patient.

9. The nasal cannula holding system of claim 1 wherein the support band is adapted to be disposed over the top of the patient's head.

10. The nasal cannula holding system of claim 1 further comprising a nose clamp adapted to hold the nose piece within the patient's nostrils.

11. A method of maintaining the position of a nasal cannula relative to the head of a patient comprising:
providing a support band and positioning the support band on the head of a patient;
providing a first fastener and attaching the first fastener to the support band on the right side of the patient's head;
providing a second fastener and attaching the second fastener to the support band on the left side of the patient's head;
providing a nasal cannula comprising a nose piece and right and left cannula tubes;
positioning the nose piece into the nostrils of the patient and the right and left cannula tubes to the respective sides of the patient's head; and
connecting the right cannula tube to the first fastener and the left cannula tube to the second fastener to secure the nasal cannula in place.

12. The method of claim 11 wherein each fastener is disposed forward and above the ears of the patient.

13. The method of claim 11 wherein each fastener is disposed behind and above the ears of the patient.

14. The method of claim 11 wherein each fastener comprises a strap with a first end and a second end, further comprising releasably fastening the first end to the second end.

15. The method of claim 14 wherein the first end is attached to the second end by a hook-and-loop fabric connection.

16. The method of claim 14 wherein the first end is attached to the second end by a snap fit connection.

17. The method of claim 14 wherein each fastener is in the form of a loop, further comprising disposing the loop of each fastener around the respective cannula tube.

18. The method of claim 11 wherein the support band is disposed around the forehead of the patient.

19. The method of claim 11 wherein the support band is disposed around the top of the patient's head.

20. The method of claim 11 wherein the support band comprises a pair of glasses.
